(19) United States
(12) Patent Application Publication
(10) Pub. No.: US 2007/0231812 A1
(43) Pub. Date: Oct. 4, 2007

(54) Method of Screening Antidiabetic Agents

(76) Inventors: Mitsuo Itakura, Tokushima (JP); Shoji Iwasaki, Ibaraki (JP); Masayasu Yoshino, Ibaraki (JP); Koichi Nishimuru, Ibaraki (JP)

Correspondence Address: SUGHRUE MION, PLLC, 2100 PENNSYLVANIA AVENUE, N.W., SUITE 800, WASHINGTON, DC, 20037, US

(21) Appl. No.: 11/587,959

(22) PCT Filed: Apr. 27, 2005
(86) PCT No.: PCT/JP05/07979

§ 371 Date: Oct. 27, 2006

(30) Foreign Application Priority Data
Apr. 28, 2004 (JP) 2004-134332

(51) Int. Cl. G01N 33/53; C12Q 1/68

(52) U.S. Cl. 435/6; 435/7.2

(57) ABSTRACT

A method of screening for an antidiabetic agent comprising (1) bringing a test substance into contact with a cell which overexpresses a polypeptide exhibiting an activity of suppressing insulin secretion under a high glucose concentration by an overexpression of the polypeptide in pancreatic β cells, and comprising an amino acid sequence of SOCS-2 or a modified or homologous amino acid sequence thereof, under a high glucose concentration, and (2) measuring an amount of insulin secreted from the cell, or (1) bringing a test substance into contact with a cell transformed with a DNA fragment comprising a human SOCS-2 promoter sequence or a modified sequence thereof, (2) measuring an amount of expressed SOCS-2, and (3) selecting a substance which suppresses the amount of expressed SOCS-2, is disclosed.

### TECHNICAL FIELD

The present invention relates to a method of screening for an antidiabetic agent.

### BACKGROUND ART

Diabetes is a disease bringing a persistent hyperglycemia, and it is considered that many environmental factors and genetic factors are the cause of diabetes. A main factor regulating blood glucose is insulin, and it is known that a deficiency of insulin or a redundant presence of various factors inhibiting the activities of insulin (such as genetic factors, lack of exercise, obesity, stress, or the like) cause hyperglycemia.

There are two major types of diabetes. These are classified into an insulin dependent diabetes mellitus (IDDM) caused by a decreased pancreatic insulin secretion due to an autoimmune disease or the like, and a noninsulin dependent diabetes mellitus (NIDDM) caused by a decreased pancreatic insulin secretion due to an exhausted pancreas with a continuous hypersecretion of insulin. It is considered that 95% or more of Japanese patients with diabetes are NIDDM, and there is a problem in that the number of such patients is increasing in accordance with changes of life-style.

In the treatment of diabetes, a diet therapy, an exercise therapy, a remedy for obesity, or the like are mainly used in mild cases, an oral medicament for diabetes (for example, an agent for promoting insulin secretion such as sulfonylureas) is administered when symptoms become severe, and an insulin preparation is administered in serious cases (non-patent references 1 and 2).

Sulfonylureas stimulate pancreatic β cells and promote insulin secretion. However, the timing of insulin secretion and an amount of insulin secreted are decided by the timing of a medicament administration and its dose, regardless of a blood glucose level. Therefore, hypoglycemia caused by a maintenance of the medicament activity, as a side effect, sometimes occurs. Further, symptoms in the digestive system such as a loss of appetite occur. Furthermore, sulfonylureas are contraindicated for patients with a hepatic or renal dysfunction or severe ketosis (non-patent reference 3).

Although the insulin preparations certainly decrease blood glucose, they must be administered by injection, and sometimes cause hypoglycemia (non-patent reference 4).

As described above, conventionally used agents for promoting insulin secretion and insulin preparations have these problems. Therefore, agents capable of an advanced control of blood glucose, i.e., agents not simply decreasing blood glucose but capable of controlling blood glucose within a normal range, are desired.

Secreted insulin transduces a signal into hepatocytes or myocytes via insulin receptors located on cell membranes thereof, and finally promotes a glucose uptake from extracellular fluid. Although intensive studies have been conducted (non-patent references 5 to 7), a detailed mechanism of an insulin signal transduction pathway has not been found.

Recently, a SOCS (suppressor of cytokine signaling) family was identified as an information transduction factor induced by various cytokines or hormones. The SOCS family commonly has an SH2 (Src homology 2) domain and a SOCS box. It is considered that the SH2 domain recognizes phosphorylated tyrosine, and suppresses signals from various cytokine receptors. It is known that sequence identities in the SOCS family are restricted to the SH2 domain and the SOCS box, that the molecular sizes vary from 579 amino acids (SOCS-7) to 198 amino acids (SOCS-2), that the SOCS family includes a molecule having a KIR (kinase inhibitory region) such as SOCS-1 or SOCS-3 and a molecule not having the KIR such as other SOCS family molecules (non-patent reference 8). The SOCS family molecules play various roles in a living body.

(non-patent reference 1) Ryuzo Abe and Masato Kasuga, “An Approach to EBM on the Treatment of Diabetes Mellitus”, Nankodo, 1997

(non-patent reference 2) Japan Diabetes Society, “Tounyoubyou chiryou gaido 2000 (Treatment of diabetes mellitus, Guide 2000)”, Bunkodo, 2000

(non-patent reference 3) “Annals of Emergency Medicine”, U.S.A., 2001, 38(1), p. 68-78

(non-patent reference 4) “Diabetes & Metabolism”, U.S.A., 1994, 20(6), p. 503-512

(non-patent reference 5) “The Journal of Clinical Investigation”, U.S.A., 2000, 106(2), p. 165-169

(non-patent reference 6) “The Journal of Biological Chemistry”, U.S.A., 1999, 274(4), p. 1865-1868

(non-patent reference 7) “Nature”, United Kingdom, 2001, 410(6831), p. 944-948

(non-patent reference 8) “Proceedings of the National Academy of Sciences of the United States of America”, U.S.A., 1998, 95, p. 114-119

### DISCLOSURE OF INVENTION

### Problems to be Solved by the Invention

Under these circumstances, the present inventors conducted an intensive search with a target of a novel antidiabetic agent which specifically promotes insulin secretion under a high glucose concentration. It is well-known that an amount of insulin secreted from pancreatic β cells is increased under a high glucose concentration, and the present inventors found that an overexpression of SOCS-2 in pancreatic β cells lowered the amount of insulin secreted under a high glucose concentration, whereas the overexpression of SOCS-2 in pancreatic β cells did not change the amount of insulin secretion under a low glucose concentration. That is, it was clarified that SOCS-2 suppresses the insulin secretion to be promoted under a high glucose concentration, from pancreatic β cells. From these findings, the present inventors found that SOCS-2 and a pancreatic β cell overexpressing SOCS-2 can be used as a screening tool for an antidiabetic agent capable of controlling blood glucose within a normal range, and constructed a screening system for a substance which increases insulin secreted from SOCS-2-overexpressing pancreatic β cells under a high glucose concentration. Further, the present inventors obtained a SOCS-2 promoter, and constructed a screening system for a substance which lowers the SOCS-2 promoter activity. As a result, the present inventors provided a novel and convenient screening method to obtain a substance useful as an agent for promoting insulin secretion (preferably an agent for specifically promoting insulin secretion under a high glucose concentration), which is an antidiabetic agent capable of controlling blood glucose within a normal range, and completed the present invention.

### Means for Solving the Problems

The present invention relates to

[1] a screening tool for an antidiabetic agent, consisting of a polypeptide exhibiting an activity of suppressing insulin secretion under a high glucose concentration by an overexpression of the polypeptide in pancreatic β cells, and comprising an amino acid sequence of SOCS-2, an amino acid sequence in which 1 to 10 amino acids are deleted, substituted, and/or added in the amino acid sequence of SEQ ID NO: 2, or an amino acid sequence having a 90% or more identity with the amino acid sequence of SEQ ID NO: 2,

[2] the screening tool of [1], wherein SOCS-2 is a human SOCS-2 or a mouse SOCS-2,

[3] a screening tool for an antidiabetic agent, consisting of a cell overexpressing the polypeptide of [1] or [2],

[4] a method of screening for an antidiabetic agent, comprising the steps of:

(1) bringing a substance to be tested into contact with the cell of [3] under a high glucose concentration, and

(2) measuring an amount of insulin secreted from the cell,

[5] the method of [4], wherein the antidiabetic agent is an agent for promoting insulin secretion,

[6] a screening tool for an antidiabetic agent, which is a DNA fragment exhibiting a human SOCS-2 promoter activity and comprising the nucleotide sequence of SEQ ID NO: 3 or a part thereof, or a nucleotide sequence or a part thereof in which 1 to 10 nucleotides are deleted, substituted, and/or added in the nucleotide sequence of SEQ ID NO: 3,

[7] a method of screening for an antidiabetic agent, comprising the steps of:

(1) bringing a substance to be tested into contact with a cell transformed with the DNA fragment of [6],

(2) measuring an amount of expressed SOCS-2, and

(3) selecting a substance which suppresses the amount of expressed SOCS-2, and

[8] the method of [7], wherein the antidiabetic agent is an agent for promoting insulin secretion.

The present invention includes a use of the screening tool of the present invention for the screening for an antidiabetic agent (preferably an agent for promoting insulin secretion, more preferably an agent for specifically promoting insulin secretion under a high glucose concentration).

For example, a human SOCS-2 (NCBI Reference Sequences No. NP_{—}003868: SEQ ID NO: 5) and a mouse SOCS-2 (NCBI Reference Sequences No. NP_{—}031732 : SEQ ID NO: 2) (identity in amino acid sequence=93%) are known, and may be used as the screening tool of the present invention. Patent references (U.S. Pat. No. 5,919,661-A, WO00/55174-A1, and WO03/039443) disclose nucleotide sequences encoding amino acid sequences having identities of 95% (189 a.a./198 a.a.), 99% (190 a.a./191 a.a.), and 100% (198 a.a./198 a.a.) with that of the human SOCS-2 consisting of 198 amino acid residues, respectively. These patent references disclose various diseases related thereto, without supports, but do not disclose or suggest an activity of inhibiting a promotion of insulin secretion under a high concentration of glucose. WO01/35732-A1 discloses a possibility that SOCS-2 controls hormone signals including growth hormone (GH) and insulin-like growth factor-I (IGF-1) and aged livestock or humans maintain their activity, a possibility of controlling obesity by regulating metabolism, a possibility of treating chronic inflammation, a possibility of preventing cardiac infarction, a fracture, or osteoporosis, and the like, but does not disclose an activity of inhibiting a promotion of insulin secretion under a high concentration of glucose. The present inventors newly found that SOCS-2 has an activity of suppressing a promotion of insulin secreted from pancreatic β cells under a high concentration of glucose, and newly revealed that SOCS-2 causes a decrease in insulin secretion under a high concentration of glucose in diabetes.

### EFFECTS OF THE INVENTION

According to the screening tool or the screening method of the present invention, a substance useful as an antidiabetic agent (preferably an agent for promoting insulin secretion, more preferably an agent for specifically promoting insulin secretion under a high glucose concentration), which can control blood glucose within a normal range.

### BRIEF DESCRIPTION OF THE DRAWINGS

FIG. 1 is a graph showing an insulin concentration (ng/mL) in each supernatant obtained by incubating MIN6B1 cells, previously infected with an adenovirus, for 20 minutes in the presence of 2.8 mmol/L or 16.8 mmol/L glucose. The abbreviation “CTRL” means a control, and the symbol “**” denotes that a significant difference against the control group was p<0.01. The vertical axis indicates the insulin concentration (ng/mL).

FIG. 2 is a graph showing an insulin concentration (ng/mL) in each supernatant obtained by incubating rat pancreatic Langerhans islets, previously infected with an adenovirus, for 1.5 hours in the presence of 2.8 mmol/L or 16.8 mmol/L glucose. The abbreviation “CTRL” means a control, and the symbol “**” denotes that a significant difference against the control group was p<0.01. The vertical axis indicates the insulin concentration (ng/mL).

FIG. 3 is a graph showing an amount of SOCS-2 mRNA measured by a quantitative PCR after incubating HepG2 cells for 3 hours in the presence or absence of IL-6, which is considered an inducer of STAT1, STAT3, or STAT5. In the vertical axis, the amount of RNA in the presence of IL-6 [IL6(+)] is shown as a relative value when the amount in the absence of IL-6 [IL6(−)] is regarded as 100.

FIG. 4 is a graph showing a reporter activity measured after incubating HepG2 cells, previously transfected with a reporter plasmid containing the SOCS-2 promoter, for 3 hours in the presence or absence of IL-6, and standardized on the basis of a co-transfected β-galactosidase activity. In the vertical axis, the activity in the presence of IL-6 [IL6(+)] is shown as a relative value when the promoter activity in the absence of IL-6 [IL6(−)] is regarded as 100.

### BEST MODE FOR CARRYING OUT THE INVENTION

Hereinafter, terms as used herein will be explained.

The term “under a high glucose concentration” or “under a high concentration of glucose” means conditions in which a glucose concentration in blood or an extracellular environment exceeds a normal range, and is preferably 16.8 mmol/L.

The term “under a low glucose concentration” or “under a low concentration of glucose” means conditions in which the above-mentioned glucose concentration is less than a normal range, and is preferably 2.8 mmol/L.

The term “pancreatic β cell” means a cell capable of secreting insulin and a matured pancreatic β cell after differentiation or regeneration, and is preferably a cell derived from mammals or an established cell line. There may be mentioned, for example, pancreatic Langerhans islets isolated from a rat or mouse pancreas, or a cell line used in studies in pancreatic β cells, such as an RIN5 cell [Proc. Natl. Acad. Sci. USA (1977) 74, 628-630], an HIT cell [Proc. Natl. Acad. Sci. USA (1981) 78, 4339-4342], an MIN6 cell [Endocrinol. (1990) 127, 126-132], an MIN6B1 cell [Endocrinol. (2003) 144, 1368-1379], a βTC cell [Endocrinol. (1990) 126, 2815-2822, Diabetes (1993) 42, 901-907], an NIT1 cell [Diabetes (1991) 40, 842-849], an INS-1 cell [Endocrinol. (1992) 130, 167-178], or a βHC cell [Mol. Cell. Biol. (1993) 13, 4223-4232].

The term “screening” includes both an identification of one or more substances having an activity of interest from many test substances, and a judgment of whether or not a substance to be tested has an activity of interest.

The present invention will be explained in detail hereinafter.

1. Screening Tool of the Present Invention

The screening tool of the present invention for an antidiabetic agent (preferably an agent for promoting insulin secretion, more preferably an agent for promoting specifically insulin secretion under a high blood concentration) includes (1) a polypeptide-type screening tool, (2) a cell-type screening tool, and (3) a promoter-type screening tool.

(1) Polypeptide-Type Screening Tool

As the polypeptide-type screening tool of the present invention, a polypeptide exhibiting an activity of suppressing insulin secretion under a high glucose concentration by an overexpression of the polypeptide in pancreatic β cells, and comprising an amino acid sequence of SOCS-2, an amino acid sequence in which 1 to 10 amino acids are deleted, substituted, and/or added in the amino acid sequence of SEQ ID NO: 2, or an amino acid sequence having a 90% or more identity with the amino acid sequence of SEQ ID NO: 2 may be used, and a human SOCS-2 or a mouse SOCS-2 is preferable.

A method of judging whether or not a polypeptide of interest exhibits the “activity of suppressing insulin secretion under a high glucose concentration by an overexpression of the polypeptide in pancreatic β cells” is not particularly limited, but the activity may be confirmed by, for example, a method described in Example 1 or Example 2.

More particularly, pancreatic β cells are transformed with an expression vector containing a DNA capable of expressing the polypeptide to be judged, and an empty vector for control, to prepare a test cell expressing the polypeptide and a control cell, respectively. After a predetermined number of hours or days (for example, 12 hours to 2 days) from the transformation, the medium is replaced with a buffer containing a high concentration or a low concentration of glucose, and the transformed cells are further incubated. After a predetermined number of minutes or hours (for example, several minutes to several hours) of incubation, an amount of insulin secreted into the buffer (i.e., a culture supernatant) is measured. When the activity of suppressing insulin secretion is observed only when the cells are stimulated by a high concentration of glucose, whereas no difference between the test cell and the control cell is observed when stimulated by a low concentration of glucose, it can be judged that the polypeptide of interest exhibits the “activity of suppressing insulin secretion under a high glucose concentration by an overexpression of the polypeptide in pancreatic β cells”.

To maintain the functions of the original polypeptide, the amino acid to be substituted is preferably an amino acid having properties similar to those of the original amino acid. For example, amino acids belonging to each of the following groups have properties similar to those of other members in the group. When these amino acids are substituted with other amino acids in the same group, the essential functions of the original protein are often maintained. Such amino acid substitution is called a conservative substitution, and is known as a method for changing an amino acid sequence while maintaining the polypeptide functions.

Nonpolar amino acids: Ala, Val, Leu, Ile, Pro, Met, Phe, and Trp

Uncharged amino acids: Gly, Ser, Thr, Cys, Tyr, Asn, and Gln

Acidic amino acids : Asp and Glu

Basic amino acids : Lys, Arg, and His

The term “identity” as used herein means a value obtained by a BLAST (Basic local alignment search tool; Altschul, S. F. et al., J. Mol. Biol., 215, 403-410, 1990). The homology in the amino acid sequence may be calculated by a BLAST search algorithm. More particularly, it may be calculated using a b12seq program (Tatiana A. Tatusova and Thomas L. Madden, FEMS Microbiol. Lett., 174, 247-250, 1999) in a BLAST package (sgi32bit edition, version 2.0.12; obtained from NCBI) in accordance with a default parameter. As a pairwise alignment parameter, a program “blastp” is used. Further, “0” as a Gap insertion cost value, “0” as a Gap elongation cost value, “SEG” as a filter for a Query sequence, and “BLOSUM62” as a Matrix are used, respectively.

The expression of SOCS-2 in a cell may be easily carried out by linking a DNA fragment encoding SOCS-2 downstream of an appropriate promoter. Such a DNA fragment may be prepared by genetic engineering techniques (for example, “Molecular Cloning”, Sambrook, J. et al., Cold Spring Harbor Laboratory Press, 1989) on the basis of sequence information, such as sequences of human SOCS-2 (SEQ ID NO: 4 and SEQ ID NO: 5) or mouse ortholog sequences corresponding thereto (SEQ ID NO: 1 and SEQ ID NO: 2).

Hereinafter, for the expression of SOCS-2, a method of obtaining a polynucleotide encoding SOCS-2, a method of preparing an expression vector for SOCS-2, and a method of preparing a cell expressing SOCS-2 will be explained.

A DNA fragment encoding SOCS-2 may be obtained by, but is not limited to, the following procedures or other known procedures (for example, “Molecular Cloning”, Sambrook, J. et al., Cold Spring Harbor Laboratory Press, 1989).

There may be mentioned, for example, (1) a method using a PCR, (2) a method using conventional genetic engineering techniques (i.e., a method of selecting a transformant containing an amino acid sequence of interest from strains transformed with a cDNA library), or a chemical synthesis method. These methods may be carried out in accordance with, for example, WO01/34785.

More particularly, SOCS-2 may be prepared in accordance with the method described in Example 1.

(2) Cell-Type Screening Tool

As the cell-type screening tool of the present invention, a cell overexpressing a polypeptide which may be used as the polypeptide-type screening tool of the present invention may be used.

A host cell, such as an eukaryotic cell or a prokaryotic cell, may be transformed with a fragment containing a polynucleotide encoding SOCS-2, by reintegrating the fragment into an appropriate vector plasmid. SOCS-2 may be expressed in a desired host cell by introducing an appropriate promoter and a sequence related to the gene expression into the vector plasmid. As the host cell, a cell which may secret insulin, preferably a pancreatic β cell, may be used.

More particularly, SOCS-2 may be expressed in a pancreatic β cell by linking a DNA fragment encoding SOCS-2 downstream of an appropriate promoter, integrating the DNA construct into an appropriate vector plasmid, and introducing the plasmid into the host cell. Alternatively, a cell in which such a construct is integrated into a chromosomal DNA may be used. As a preferable embodiment, there may be mentioned, for example, a method described in Example 1 or Example 2.

Transformation of a host cell for gene expression may be carried out, for example, by using a commonly used lipofectamine agent.

(3) Promoter-Type Screening Tool

As the promoter-type screening tool of the present invention, a DNA fragment exhibiting a human SOCS-2 promoter activity and comprising the nucleotide sequence of SEQ ID NO: 3 or a part thereof, or a nucleotide sequence or a part thereof in which 1 to 10 nucleotides are deleted, substituted, and/or added in the nucleotide sequence of SEQ ID NO: 3 may be used.

Such a DNA fragment may be obtained by conventional genetic engineering techniques, for example, a method described in Example 3.

The term “human SOCS-2 promoter activity” as used herein means a promoter activity of a human SOCS-2 gene, more specifically, a promoter activity of a DNA consisting of the nucleotide sequence of SEQ ID NO: 3. A method of judging whether or not a DNA of interest exhibits the “human SOCS-2 promoter activity” is not particularly limited, but the activity may be confirmed by a known conventional method, for example, by linking an appropriate reporter gene at the 3′-downstream of the DNA to be judged, introducing the DNA construct into a eukaryotic cell (preferably an animal cell strain), cultivating the cell, and measuring an amount of the reporter gene expressed in the cell. More specifically, the activity may be confirmed, for example, by a method described in Example 3.

2. Screening Method of the Present Invention

The present inventors found that the SOCS-2 gene has an activity of suppressing an amount of insulin secreted from pancreatic β cells under a high glucose concentration when overexpressed in pancreatic β cells. Therefore, a method of screening for an antidiabetic agent (preferably an agent for promoting insulin secretion, more preferably an agent for specifically promoting insulin secretion under a high glucose concentration) using, as an index, an amount of insulin secreted from pancreatic β cells overexpressing SOCS-2, a change in an amount of SOCS-2 expressed, or a quantitative or qualitative change in binding to a molecule to which SOCS-2 binds for transducing a signal or performing its function in pancreatic β cells, may be constructed.

Test substances which may be used in the screening method of the present invention are not particularly limited, but there may be mentioned, for example, commercially available compounds (including peptides), various known compounds (including peptides) registered in chemical files, compounds obtained by combinatorial chemistry techniques [N. Terrett et al., Drug Discov. Today, 4(1):41,1999], culture supernatants of microorganisms, natural components derived from plants or marine organisms, animal tissue extracts, or compounds (including peptides) obtained by chemically or biologically modifying compounds (including peptides) selected by the screening method of the present invention.

The screening method in the present invention includes, but is not limited to, the following methods.

(1) Screening Method Using the Measurement of an Amount of Insulin Secreted

Pancreatic β cells are transformed with a DNA capable of expressing SOCS-2 to prepare a test cell expressing SOCS-2. After a predetermined number of hours or days (for example, 12 hours to 2 days) from the transformation, the medium is replaced with a buffer containing a predetermined concentration of glucose, and the transformed cells are further incubated. After a predetermined number of minutes or hours (for example, several minutes to several hours) of incubation, an amount of insulin secreted into the buffer (i.e., a culture supernatant) is measured. During the incubation, a test substance is added, and not added, in the glucose-containing buffer to treat, and not treat, the test cells, respectively. After the incubation, insulin secreted from the test cells into the culture supernatant is measured. In this procedure, it is preferable that insulin secretion is significantly suppressed by the expression of SOCS-2 in pancreatic β cells, and that the suppression of insulin secretion is significantly recovered to the normal level or promoted by the treatment of the test substance. The normal level means an amount of insulin secreted from control cells transformed with an empty vector without SOCS-2 and cultivated under a high concentration of glucose (i.e., control cells in which insulin secretion is not suppressed). Further, it is preferable that no difference between an amount of insulin secreted when treated with the test substance and that when not treated therewith is observed under a low concentration of glucose. For example, a method described in Example 1 or Example 2 is preferable. Significant suppression or recovery of insulin secretion may be judged, for example, by Student's t-test on the basis of amounts of insulin secreted from the test cell group and the control cell group. When the significant difference value of the test cell to the control cell is p<0.05 (preferably p<0.01), it may be decided that the change is significant.

Hereinafter, a method of measuring an amount of secreted insulin will be explained in accordance with concrete examples.

A cell which may be used as the cell-type screening tool of the present invention may be cultivated in accordance with a conventional method. A medium used in the cultivation may be appropriately selected from commonly used various media in accordance with a host cell to be used. For example, when the above-mentioned MIN6 cell is used as a host, a Dulbecco modified Eagles' medium (DMEM) supplemented with 10% serum components such as fetal bovine serum (FBS) may be used.

An amount of secreted insulin may be measured by once washing pancreatic β cells expressing SOCS-2, cultivating the cells under a high or low concentration of glucose for several hours, and measuring the concentration of insulin contained in a culture supernatant. The concentration of insulin may be measured, for example, by using a commercially available kit for measuring an insulin concentration, as described in Examples, in accordance with a protocol attached thereto.

(2) Screening Method Using Suppression of SOCS-2 Expression as an Index

The overexpression of SOCS-2 in pancreatic β cells suppresses insulin secretion under a high glucose concentration, and therefore, an antidiabetic agent (preferably an agent for promoting insulin secretion, more preferably an agent for specifically promoting insulin secretion under a high glucose concentration) may be identified by using suppression of the SOCS-2 expression as an index. Screening may be carried out, for example, by analyzing an amount of endogenous SOCS-2 expressed in pancreatic β cells, to select an antidiabetic agent (preferably an agent for promoting insulin secretion, more preferably an agent for specifically promoting insulin secretion under a high glucose concentration). Alternatively, screening may be carried out by preparing an expression vector in which a promoter region of SOCS-2 is linked upstream of an appropriate reporter gene (such as a luciferase gene), bringing test compounds into contact with a cell transformed with the expression vector, and analyzing a change in expression of the reporter gene.

Hereinafter, the above screening methods will be explained in accordance with concrete examples.

RNAs may be prepared from pancreatic β cells untreated or treated with a test substance in accordance with a conventional method. The obtained RNA preparation may be subjected to an agarose gel electrophoresis in accordance with a known method, and the separated RNAs transferred to a nitrocellulose membrane. The membrane may be subjected to Northern blotting using a labeled oligo-DNA probe containing a partial nucleotide sequence of SOCS-2, to detect a change in an amount of expressed RNA having a SOCS-2 nucleotide sequence, due to the test substance. As a result, a substance which suppresses an amount of expressed SOCS-2 may be selected from a population of test substances.

Proteins may be prepared from pancreatic β cells untreated or treated with a test substance in accordance with a conventional method. The obtained protein preparation may be subjected to a protein electrophoresis in accordance with a known method, and the separated proteins transferred to a polyvinylidene fluoride (PVDF) membrane. The membrane may be subjected to Western blotting using an antibody specific to SOCS-2, to detect a change in an amount of expressed SOCS-2 polypeptide, due to the test substance. As a result, a substance which suppresses an amount of expressed SOCS-2 polypeptide may be selected from a population of test substances.

A change in an amount of expressed RNA having a SOCS-2 nucleotide sequence due to the test substance may be quantitatively detected by a real-time PCR method using an oligo-DNA primer containing a partial nucleotide sequence of SOCS-2. Specifically, the real-time PCR may be carried out in accordance with a method described in Example 4. As a result, a substance which suppresses an amount of expressed SOCS-2 may be selected from a population of test substances.

Screening may be carried out by preparing an expression vector in which a promoter region of SOCS-2 is linked upstream of an appropriate reporter gene (such as a luciferase gene), bringing test compounds into contact with a cell transformed with the expression vector, and analyzing a change in expression of the reporter gene. As a result, a substance capable of controlling the promoter activity, that is, a substance capable of controlling the SOCS-2 activity directly or indirectly, may be obtained. Specifically, a method described in Example 3 is preferable. As a substance capable of suppressing the promoter activity, a substance capable of significantly suppressing the reporter activity, in comparison with an activity when the promoter is activated, is preferable.

According to the screening tool or the screening method of the present invention, a substance useful as an antidiabetic agent (preferably an agent for promoting insulin secretion, more preferably an agent for specifically promoting insulin secretion under a high glucose concentration) capable of controlling blood glucose within a normal range can be selected, and the selected substance has an effect of treating diabetes (preferably an effect of promoting insulin secretion, more preferably an effect of specifically promoting insulin secretion under a high glucose concentration). The effect of the selected substance on the treatment of diabetes may be confirmed by a known method, for example, an assay system using a diabetes model animal.

The effect of specifically promoting insulin secretion under a high glucose concentration may be judged by confirming that an amount of insulin secreted is significantly increased under a high glucose concentration in comparison with a control group, and that an increase in insulin secretion of a group treated with a test compound in comparison with the control group under a high glucose concentration is significantly (preferably 1.5 times or more, more preferably 3 times or more) increased in comparison with that under a low glucose concentration. Whether or not an amount of insulin secreted in the group treated with a test compound is significantly increased in comparison with that in the control group may be judged by, for example, Student's t-test on the basis of the following experiment. When an amount of insulin secreted is increased in the group treated with a test compound, and the significant difference value thereof to the control group is p<0.05, preferably p<0.01, it may be decided that an amount of insulin secreted is significantly increased.

Experiment of Secreted Insulin Using Mouse Pancreatic β Cell Line MIN6B1

MIN6B1 cells (2×10⁵ cells) are seeded on a 24-well plate, and cultured for 24 hours in 0.5 mL of a DMEM (GIBCO) containing 10% fetal bovine serum (SIGMA). The medium is aspirated, and the cells are washed with KRB-HEPES (140 mmol/L NaCl, 3.6 mmol/L KCl, 0.5 mmol/L NaH₂PO₄, 0.5 mmol/L MgSO₄, 1.5 mmol/L CaCl₂, 10 mmol/L Hepes, 2 mmol/L NaHCO₃, 0.1% BSA, pH 7.4). After 2.8 mmol/L glucose-containing KRB-HEPES (1 mL) is added, the whole is incubated at 37° C. for 30-60 minutes in the presence of 5% CO₂.

After the buffer is aspirated, a test compound is added in the form of a solution (0.5 mL) prepared by diluting the compound with 2.8 mmol/L or 16.8 mmol/L glucose-containing KRB-HEPES, and the whole is incubated at 37° C. for 20 minutes in the presence of 5% CO₂. A supernatant is used for measuring an amount of insulin secreted.

A commercially available insulin radioimmunoassay kit (Rat insulin [125I] RIA system; Amersham Bioscience) may be used for the measurement of the amount of insulin secreted.

When the stimulation by the test compound does not cause an increase of an amount of insulin secreted in the presence of 2.8 mmol/L glucose, but causes an increase of an amount of insulin secreted in the presence of 16.8 mmol/L glucose, it may be confirmed that the test compound exhibits an activity of promoting insulin secretion only when stimulated by a high concentration of glucose.

### EXAMPLES

The present invention now will be further illustrated by, but is by no means limited to, the following Examples. The procedures were performed in accordance with the known methods (Maniatis, T., et al., “Molecular Cloning—A Laboratory Manual”, Cold Spring Harbor Laboratory, NY, 1982), unless otherwise specified.

### Example 1

### Measurement of Insulin Secreted from MIN6B1 Cells Overexpressing SOCS-2

(1) Preparation of SOCS-2-Overexpressing Virus Using Adenovirus Vector

On the basis of NCBI Reference Sequences No. NM_{—}007706, synthetic oligo-DNAs having a KpnI or XhoI recognition site at the terminus [5′-CGGGGTACCGCCATGACCCTGCGGTGCCTGGAGCCCTCC-3′ (SEQ ID NO: 6) and 5′-CCGCTCGAGTTATACCTGGAATTTATATTCTTCCAA-3′ (SEQ ID NO: 7)] were designed. A PCR was carried out using these synthetic oligo-DNAs to obtain a DNA fragment (SEQ ID NO: 1) encoding a mouse SOCS-2 consisting of the amino acid sequence of SEQ ID NO: 2. In the PCR, a Pyrobest DNA polymerase (TAKARA) was used. A reaction at 94° C. for 1 minute was carried out, a cycle composed of reactions at 98° C. for 5 seconds and at 68° C. for 1 minute was repeated 5 times, a cycle composed of reactions at 98° C. for 5 seconds and at 65° C. for 1 minute was repeated 5 times, a cycle composed of reactions at 98° C. for 5 seconds, at 60° C. for 30 seconds, and at 72° C. for 1 minute was repeated 30 times, and a reaction at 72° C. for 1 minute was carried out. The resulting DNA fragment was digested with restriction enzymes KpnI and XhoI, and inserted into the multicloning site (KpnI/XhoI) of an adenovirus vector pAdTrack-CMV (Tong-Chuan He et al., Proc. Natl. Acad. Sci, USA, vol. 95, pp. 2509-2514, 1998) to obtain a SOCS-2/pAdTrack-CMV vector.

In accordance with a known protocol [“A Practical Guide for Using the AdEasy System” (http://www.coloncancer.org/adeasy.htm “http://www.coloncancer.org/adeasy/protocol2.htm”)], a liquid of a high-titer adenovirus expressing SOCS-2 was prepared. As an adenovirus for control, pAdTrack-CMV was used.

In this connection, an absorbance at 260 nm (A260) was measured and converted into an amount of virus in accordance with the following equation:
1 A260=1.1×10¹² virus particles =3.3×10¹¹ pfu/mL
(2) Preparation of Pancreatic β Cells Overexpressing SOCS-2 by Adding SOCS-2-Expressing Adenovirus to Mouse Pancreatic β Cell Line MIN6B1

Mouse pancreatic β cell line MIN6B1 cells [Endocrinol. (2003) 144,. 1368-1379] were infected with SOCS-2/pAdTrack-CMV or pAdTrack-CMV (control) to prepare pancreatic β cells overexpressing SOCS-2, as described below.

MIN6B1 cells (2×10⁵ cells) were seeded on a 24-well plate, and cultured for 24 hours in 0.5 mL of a minimum essential medium DMEM (GIBCO) containing 10% fetal bovine serum (SIGMA). SOCS-2/pAdTrack-CMV or pAdTrack-CMV (control) was added to each medium at a concentration of 4×10⁸ pfu/well.

Infection of pancreatic β cells with each adenovirus was confirmed by visually observing a fluorescence from GFP (green fluorescent protein) contained in pAdTrack-CMV under a fluorescent microscope. Expression of SOCS-2 was confirmed by Western blotting using a commercially available antibody specific to SOCS-2 (anti-SOCS-2 antibody; ANASPEC Incorporated, catalogue code 28133) as the first antibody and a rabbit IgG-HRP (horseradish peroxidase) conjugate antibody (Biorad) as the second antibody.

(3) Measurement of Insulin Secreted in Cells Expressing SOCS-2

After 14 hours from the adenovirus infection of pancreatic β cells, each medium was aspirated, and cells were washed three times with KRB-HEPES (140 mmol/L NaCl, 3.6 mmol/L KCl, 0.5 mmol/L NaH₂PO₄, 0.5 mmol/L MgSO₄, 1.5 mmol/L-CaCl₂, 10 mmol/L Hepes, 2 mmol/L NaHCO₃, and 0.1% BSA, pH 7.4), and incubated in 2.8 mmol/L glucose-containing KRB-HEPES (1 mL) at 37° C. for 1 hour in the presence of 5% CO₂.

After the buffer was aspirated, 2.8 mmol/L or 16.8 mmol/L glucose-containing KRB-HEPES was added. The cells were further incubated at 37° C. for 20 minutes in the presence of 5% CO₂, and an amount of insulin secreted into the supernatant was measured.

A commercially available insulin radioimmunoassay kit (Rat insulin [125I] RIA system; Amersham Bioscience) was used for the measurement of the amount of insulin secreted.

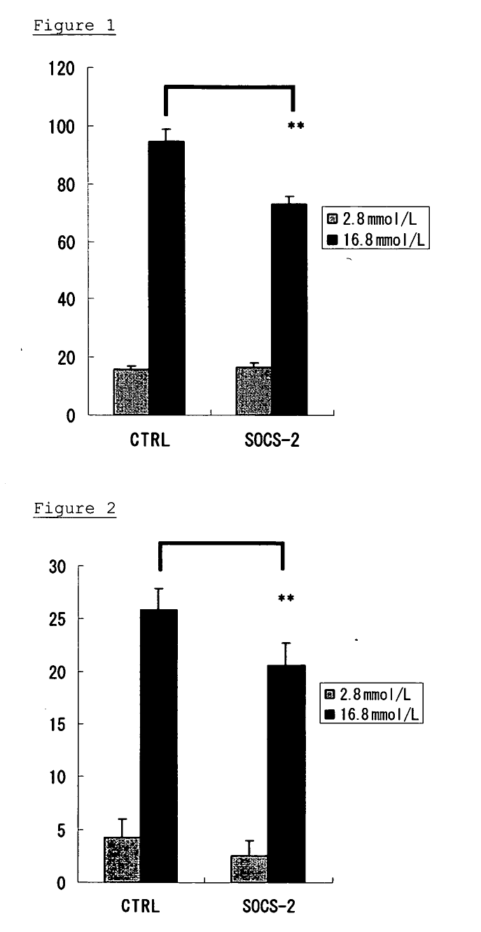

As shown in FIG. 1, the expression of SOCS-2 inhibited the activity of promoting insulin secretion only when stimulated with a high concentration (16.8 mmol/L) of glucose significantly in comparison with the control, whereas no difference was observed when stimulated with a low concentration (2.8 mmol/L) of glucose.

It was found from the result that SOCS-2 acts as an aggravating factor in diabetes by inhibiting insulin secretion into cells by the overexpression of SOCS-2. The symbol “**” in FIG. 1 denotes that a significant difference against the control group was p<0.01 (Student's t-test).

### Example 2

### Experiment of Secreting Insulin Using Rat Pancreatic Langerhans Islets Overexpressing SOCS-2

(1) Isolation of Rat Pancreatic Langerhans Islets

After 4 male rats (6-8 weeks old, 350 to 450 g in weight) were anesthetized, the abdomen was incised and exposed. A bile duct of the liver was ligated, and blood was drawn from the heart. From the bile duct, 5 mL of HBSS-HEPES (136.8 mmol/L NaCl, 5.3 mmol/L KCl, 0.8 mmol/L MgSO₄, 1 mmol/L Na₂HPO₄, 0.44 mmol/L KH₂PO₄, 4.1 mmol/L NaHCO₃, 10 mmol/L Hepes, 1 mmol/L CaCl₂, and 2 mmol/L glucose, pH 7.2) supplemented with 0.02% Liberase (Roche Diagnostic) was injected using a winged needle. The pancreas was removed and transferred to a tube containing 5 mL of HBSS-HEPE, and incubated at 37° C. for 20 minutes. After the incubation, the whole was stirred, and ice-cold HBSS-HEPES-0.35% BSA was added. The mixture was centrifuged at 1500 rpm for 1 minute to remove the supernatant. To the precipitated tissues, 10 mL of HBSS-HEPES-0.35% BSA was added, and the tissues were suspended using a needle. This washing step was repeated twice. After the centrifugation, the supernatant was removed, and 10 mL of a 8.3% Ficoll-Conray solution was added and suspended. Further, 10 mL of HBSS-HEPES-0.35% BSA was added, and the whole was centrifuged for 20 minutes. After the centrifugation, pancreatic Langerhans islets between two liquid layers were collected. To the collected pancreatic Langerhans islets, 10 mL of HBSS-HEPES-0.35% BSA was added, and the whole was centrifuged. The pancreatic Langerhans islets were seeded on a 6-well plate at a concentration of 60 cells per well, and incubated in 2 mL of RPMI1640 (Invitrogen) supplemented with 10% fetal bovine serum (SIGMA) for 1 day.

(2) Preparation of Rat Pancreatic Langerhans Islets Overexpressing SOCS-2 by Adding SOCS-2-Expressing Adenovirus to Rat Pancreatic Langerhans Islets

SOCS-2/pAdTrack-CMV or pAdTrack-CMV (control) was added to each medium at a concentration of 1.2×10¹⁰ pfu. The infection of pancreatic Langerhans islets with each adenovirus and the expression of SOCS-2 were confirmed by the methods described in Example 1(2).

(3) Measurement of Insulin Secreted from Pancreatic Langerhans Islets Overexpressing SOCS-2

After 42 hours from the addition of each adenovirus to the isolated pancreatic Langerhans islets, each medium was aspirated, and a washing treatment with KRB-HEPES-BSA (140 mmol/L NaCl, 5 mmol/L KCl, 1.2 mmol/L KH₂PO₄, 1.2 mmol/L MgSO₄, 1.7 mmol/L CaCl₂, 5.3 mmol/L NaHCO₃, 10 mmol/L Hepes, and 0.5% BSA, pH7.4) containing 2.8 mmol/L glucose was carried out three times. To each 1.5-mL tube, 5 pancreatic Langerhans islets were transferred, and incubated in 500 μL of KRB-HEPES-BSA containing 2.8 mmol/L glucose at 37° C. for 30 minutes.

Further, KRB-HEPES-BSA containing glucose was added so that the final concentration of glucose became 2.8 mmol/L or 16.8 mmol/L, and the pancreatic Langerhans islets were incubated for 90 minutes. Each supernatant was used to measure an amount of insulin secreted.

A commercially available insulin radioimmunoassay kit (Rat insulin [125I] RIA system; Amersham Bioscience) was used for the measurement of the amount of insulin secreted.

As shown in FIG. 2, the expression of SOCS-2 inhibited the activity of promoting insulin secretion only when stimulated with a high concentration (16.8 mmol/L) of glucose significantly in comparison with the control, whereas no difference was observed when stimulated with a low concentration (2.8 mmol/L) of glucose.

It was found from the result that SOCS-2 acts as an aggravating factor in diabetes by inhibiting insulin secretion into cells by the overexpression of SOCS-2. The symbol “**” in FIG. 2 denotes that a significant difference against the control group was p<0.01 (Student's t-test).

### Example 3

### Measurement of Promoter Activity of SOCS-2 Using Reporter System With SOCS-2 Promoter

(1) Construction of Reporter Vector of SOCS-2 Promoter Region

On the basis of the registered sequence (NCBI Reference Sequences No. NM_{—}003877) of human SOCS-2, a genomic sequence which accords with the sequence was specified as the sequence of NCBI GenBank accession No. NC_{—}000012.5_{—}93000001_{—}94000000. To obtain a DNA consisting of nucleotides 893,596-898,678 of NC_{—}000012.5_{—}93000001_{—}94000000, which was considered the promoter region of human SOCS-2, i.e., a DNA consisting of the nucleotide sequence of SEQ ID NO: 3, the first PCR was carried out using synthetic oligo DNAs [5′-gtgACGCGTGCTCCCTCCAAGTGGTGGAAAAGTTGA-3′ (SEQ ID NO: 8) and 5′-gtgGCTAGCGCGCTGCGGAAAATGCAAACCACCAAC-3′ (SEQ ID NO: 9)] having restriction enzyme sites NluI and NheI, respectively, and an LA Taq (TAKARA; catalogue code RR002A). The resulting PCR product was diluted to 1/50 with sterile water, and the second PCR was carried out using the diluted solution as a template, synthetic oligo DNAs [5′-gtgACGCGTGACCTGTATGGTCATTATCACTCATCA-3′ (SEQ ID NO: 10) and 5′-gtgGCTAGCGCGCTCTTACCTCGACCTCGGCCGCG-3′ (SEQ ID NO: 11)], and an LA Taq (TAKARA; catalogue code RR002A). In the first PCR, a reaction at 94° C. for 1 minute was carried out, a cycle composed of reactions at 98° C. for 10 seconds and at 72° C. for 5 minutes was repeated 5 times, and a cycle composed of reactions at 98° C. for 10 seconds and at 68° C. for 5 minutes was repeated 5 times. In the second PCR, a cycle composed of reactions at 98° C. for 10 seconds and at 68° C. for 5 minutes was repeated 10 times, a cycle composed of reactions at 98° C. for 10 seconds and at 65° C. for 5 minutes was repeated 25 times, and a reaction at 72° C. for 5 minutes was carried out. The obtained DNA fragment of approximately 5 kb was sequenced using a DNA sequence reagent (BigDye3.1; Applied Biosystems) and a DNA sequencer (model PRISM3700; Applied Biosystems) in accordance with protocols attached thereto, to confirm that it was an expected upstream sequence containing an exon of SOCS-2 of NCBI GenBank accession No. NT_{—}019546. The obtained DNA fragment contained plural sequences (TTCCCRKAA; STATx, TRANSFAC accession No. M00223) characteristic of a STAT binding. In this connection, it is predicted that STAT1, STAT3, and STAT5 bind to this characteristic sequence, and it is considered that SOCS-2 reacts with STAT1, STAT3, and STAT5. The DNA fragment was inserted between the MluI and Nhe sites of PGVB-2 (PicaGene basic vector 2; Toyo Ink MFG) to construct a SOCS-2 reporter vector.

(2) Measurement of SOCS-2 Promoter Activity

First, an endogenous SOCS-2 promoter activity was measured. Since a stimulation capable of promoting the SOCS-2 promoter activity in pancreatic β cells was unknown, an amount of SOCS-2 mRNA was measured when stimulated by IL-6, which was a typical stimulation capable of increasing a transcriptional activity of STAT1, STAT3, or STAT5. It is considered that STAT1, STAT3, and STAT5 transduce an external signal, and finally promote a SOCS-2 transcriptional activity. More particularly, human HepG2 cells in which the IL-6 reactivity was confirmed were used to measure and compare amounts of endogenous SOCS-2 mRNA expressed when stimulated with or without IL-6 stimulation. The amount of gene expressed was compensated by measuring an amount of a G3PDH (Glyceraldehyde 3-phosphate dehydrogenase) gene expressed, at the same time. As a measuring system, a PRISM TM 7700 Sequence Detection System (Applied Biosystems) and a SYBR Green PCR Master Mix (Applied Biosystems). In this measuring system, an amount of fluorescence from a SYBR Green I dye incorporated into double-stranded DNAs amplified by PCR was detected and quantified in real time, to determine the amount of gene expressed.

The measurement was carried out in accordance with the following procedure.

HepG2 cells (ATCC accession No. HB-8065) were-seeded on a 6-well plate, incubated in 2 mL of a minimum essential medium DMEM (GIBCO) containing 10% fetal bovine serum (SIGMA) for 1 day, and further incubated in the presence of IL-6 (10 ng/mL; R&D Systems) for 3 hours. Total RNAs were prepared using an RNA extraction reagent (RNeasy; Qiagen) in accordance with a protocol attached thereto. A kit for reverse transcription (Advantage™ RT-for-PCR Kit; Clonetech) and 0.25 μg of the total RNAs were used to carry out reverse transcription from the total RNAs to single-stranded cDNA in a system of 20 μL. In this reaction, a combination of synthetic oligo DNAs having 5′-CCTTTATCTGACCAAACCGCTCTA-3′ (SEQ ID NO: 12) and 5′-TGTTAATGGTGAGCCTACAGAGATG-3′ (SEQ ID NO: 13) for the SOCS-2 gene, and a combination of synthetic oligo DNAs having 5′-CCTGACCTGCCGTCTAGAAAA-3′ (SEQ ID NO: 14) and 5′-CGCCTGCTTCACCACCTT-3′ (SEQ ID NO: 15) for the G3PDH gene were used. The real time measurement of PCR amplification by a PRISM TM 7700 sequence detection system (Applied Biosystems) was carried out in accordance with a protocol attached thereto. In each system, 5 μL of single-stranded cDNA, 12.5 μL of 2×SYBR green reagent, and 7.5 pmol of each primer were used. A calibration curve was prepared by using 5 μL of solutions prepared by appropriately diluting 0.1 μg/μL mouse genomic DNA (Clonetech) instead of the single-stranded cDNA. In the PCR, reactions at 50° C. for 10 minutes and at 95° C. for 10 minutes were carried out, and a cycle composed of reactions at 95° C. for 15 seconds and at 60° C. for 60 seconds was repeated 45 times.

The amount of mouse SOCS-2 gene expressed was compensated with that of the G3PDH gene expressed in accordance with the following equation:
C=A/B
[C: Compensated amount of SOCS-2 expressed, A: Measured value of the SOCS-2 gene expressed, and B: Measured value of the G3PDH gene expressed]

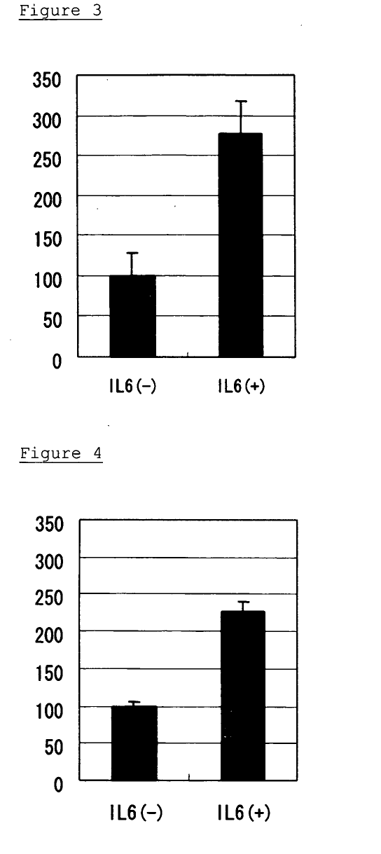

As shown in FIG. 3, it was found that the expression of the endogenous SOCS-2 gene was increased by a factor of approximately 2, by the IL-6 stimulation.

(3) Measurement of SOCS-2 Promoter Activity by Reporter

HepG2 cells (ATCC accession No. HB-8065) was transformed with the SOCS-2 reporter vector or the control vector prepared in Example 3(1) to measure the activity when stimulated with or without IL-6. More particularly, HepG2 cells were seeded on a 6-well plate, and incubated in 2 mL of a minimum essential medium DMEM (GIBCO) containing 10% fetal bovine serum (SIGMA) for 1 day. The cells were transformed with each plasmid vector (0.2 μg) using FuGENE™ 6 (BOEHRINGER MANNHEIM, USA; 1814 443), together with plasmid pCH110 (0.2 μg) containing a β-galactosidase gene controlled by a β actin promoter to standardize the efficiency of gene introduction. After 8 hours from the transformation, the cells were stimulated with IL-6, and incubated for 3 hours. The cells were lysed with a cell lysis solution LCβ (Toyo Ink MFG), and a luciferase activity was measured using a PicaGene coloring kit (Toyo Ink MFG; 309-04321).

The result is shown in FIG. 4. Each reporter activity was standardized by the β-galactosidase activity, and shown as a relative value when the value in the absence of IL-6 was regarded as 100. The expression of the SOCS-2 gene was increased by a factor of approximately 2, by the IL-6 stimulation.

This result that the expression of the reporter system was increased by a factor of approximately 2, by the IL-6 stimulation accords with the result obtained in Example 3(2) that the expression of the endogenous SOCS-2 gene was increased by a factor of approximately 2, by the IL-6 stimulation, and therefore, it was confirmed that the sequence obtained in this Example was the promoter of the SOCS-2 gene.

### INDUSTRIAL APPLICABILITY

SOCS-2 expressed in pancreatic β cells has an activity of inhibiting insulin secretion in pancreatic β cells under a high concentration of glucose. Therefore, pancreatic β cells overexpressing SOCS-2 may be used to construct a convenient screening system for identifying a substance useful as an antidiabetic agent capable of controlling blood glucose within a normal range (preferably an agent for promoting insulin secretion, more preferably an agent for specifically promoting insulin secretion under a high glucose concentration). Further, according to the screening method of the present invention using the promoter sequence, an antidiabetic agent capable of suppressing a SOCS-2 induction may be identified efficiently.

According to the screening method of the present invention, a pharmaceutical composition for treating diabetes capable of controlling blood glucose within a normal range (preferably a pharmaceutical composition for promoting insulin secretion, more preferably a pharmaceutical composition for specifically promoting insulin secretion under a high glucose concentration) may be prepared.

Although the present invention has been described with reference to specific embodiments, various changes and modifications obvious to those skilled in the art are possible without departing from the scope of the appended claims.

### FREE TEXT IN SEQUENCE LISTING

Features of “Artificial Sequence” are described in the numeric identifier <223>in the Sequence Listing. Each of the nucleotide sequences of SEQ ID NOS: 6 to 15 is an artificially synthesized primer sequence.

1. A screening tool for an antidiabetic agent, consisting of a polypeptide exhibiting an activity of suppressing insulin secretion under a high glucose concentration by an overexpression of the polypeptide in pancreatic β cells, and comprising an amino acid sequence of SOCS-2, an amino acid sequence in which 1 to 10 amino acids are deleted, substituted, and/or added in the amino acid sequence of SEQ ID NO: 2, or an amino acid sequence having a 90% or more identity with the amino acid sequence of SEQ ID NO: 2.

2. The screening tool according to claim 1, wherein SOCS-2 is a human SOCS-2 or a mouse SOCS-2.

3. A screening tool for an antidiabetic agent, consisting of a cell overexpressing the polypeptide of claim 1.

4. A method of screening for an antidiabetic agent, comprising the steps of:
(1) bringing a substance to be tested into contact with the cell of claim 3 under a high glucose concentration, and
(2) measuring an amount of insulin secreted from the cell.

5. The method according to claim 4, wherein the antidiabetic agent is an agent for promoting insulin secretion.

6. A screening tool for an antidiabetic agent, which is a DNA fragment exhibiting a human SOCS-2 promoter activity and comprising the nucleotide sequence of SEQ ID NO: 3 or a part thereof, or a nucleotide sequence or a part thereof in which 1 to 10 nucleotides are deleted, substituted, and/or added in the nucleotide sequence of SEQ ID NO: 3.

7. A method of screening for an antidiabetic agent, comprising the steps of:
(1) bringing a substance to be tested into contact with a cell transformed with the DNA fragment of claim 6,
(2) measuring an amount of expressed SOCS-2, and
(3) selecting a substance which suppresses the amount of expressed SOCS-2.

8. The method according to claim 7, wherein the antidiabetic agent is an agent for promoting insulin secretion.

9. A screening tool for an antidiabetic agent, consisting of a cell overexpressing the polypeptide of claim 2.

10. A screening tool for an antidiabetic agent, consisting of a cell overexpressing the polypeptide of claim 9.

11. A method of screening for an antidiabetic agent, comprising the steps of:
(1) bringing a substance to be tested into contact with the cell of claim 10 under a high glucose concentration, and
(2) measuring an amount of insulin secreted from the cell.

12. The method according to claim 11, wherein the antidiabetic agent is an agent for promoting insulin secretion.


SEQUENCE LISTING (15 sequences)

SEQ ID NO: 1 - DNA, 594 bp, Mus sp.
  CDS (1)..(594)
atgaccctgcggtgcctggagccctccgggaatggagcggacaggacgcggagccagtgggggaccgcggggttgccggaggaacagtcccccgaggcggcgcgtctggcgaaagccctgcgcgagctcagtcaaacaggatggtactggggaagtatgactgttaatgaagccaaagagaaattaaaagaggctccagaaggaactttcttgattagagatagttcgcattcagactacctactaactatatccgttaagacgtcagctggaccgactaacctgcggattgagtaccaagatgggaaattcagattggattctatcatatgtgtcaagtccaagcttaaacagtttgacagtgtggttcatctgattgactactatgtccagatgtgcaaggataaacggacaggcccagaagccccacggaatgggactgttcacctgtacctgaccaaacctctgtatacatcagcacccactctgcagcatttctgtcgactcgccattaacaaatgtaccggtacgatctggggactgcctttaccaacaagactaaaagattacttggaagaatataaattccaggta

SEQ ID NO: 2 - protein, 198 aa, Mus sp.
MTLRCLEPSGNGADRTRSQWGTAGLPEEQSPEAARLAKALRELSQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPTLQHFCRLAINKCTGTIWGLPLPTRLKDYLEEYKFQV

SEQ ID NO: 3 - DNA, 6424 bp, Homo sapiens
catacaatgctcaaggcagacttttatttctcagggttatcttaataaagaccttccactcccttagaacaagtgttctcacctgaggtcaacagacccatgcggatttagagtgaggtgtcaggttgtcaatcaagcccccaaatctgtgcatctagaacatttttctgcagagaaagtcttaggtgtagcctgccaataaatgcctgcagagctaggtgggaatatagaagaatgaaatacatcaggtgtgagaacccagtctcttaaacacagaggaatattattttcttccacagagaaatatgccttctcccttttattctgacatattcagccttctgggtctgtcttttatttcgtcacttctggtagagacttgggtccaaataaatatctctccaccataagaaacagtaatgcaagggtgatgacaaaaggcaagcaatgctcagtgttggggagaccttggggcgggtgaggattgggcaaacgggagggcacatgccttctaaggcaggcactcactcctctgcttcagttgatcactgccttgaggaaatgggacccagtattgacagaccttatggctgattaagagaaagcagaaatctggttctctttggagatgaaatctcaatttagttcacattttctttagagaatcctggcataccaaccagaacactctgagtgagtcaaatatggtcccgataggcaggtggtttgtgacctctggtctctagattctaccggaatttctgattgattcatgtcttaaataagattgatgactgctgtcctagaacatgtttcccctccattctgtgggcggaaagacagacggcatggacatttaccatgttcttgggtcccagaagtccaggagaaagccatctgcctcactgctccctgccccatcctgggcccagagcactaatgccagtggcatcagctctgctcctctccctcttcctttgtctaccagtgagtgtcctccattatggggatggagcaagcagtatattgtaaaagggacttagtgctgagaagcaccagaggaaaagtaggagatgctcattaaagtggaaggcagctctcttcttccccttctcaattctgcattctgctctctcccctaaatgccccccacccaacccccaaactcttgagaatgcccaaatccaaccaggtcaggaaaaaataaactcactggctcctgtattgagaagtccaagtgtggtgcggcttccggcatgtgggattcaagcactcaggaatttctccctctcacccaacctcagctctgctctcttctctgttgcctttgttctttgacaggctccctccaagtggtggaaaagttgacctgtatggtcattatcactcatcatttcagaaggggagagcctgcttctcttgtagcatttccatcaaatcctgaaaataaaaataatctggttggttctactggacttgtgatactggacaaacctctatgtccaagagctcgagtcattcccattggctcacctgggccacacactcttcgttgagtgcatcccaggatcacagggagtcttgagggtgctgtgtgtggtggtggtggtggtacggagatggcaaactctaaaaggaagagatggtggatacttaaagcctaccttctgcccacaggtgtgtcagacacaatctccaaaggccatcctattgttaccctatcccccaaacaaacactgaaaaactctcctggcaatttggcagtagctcagtgtggagcatttgttaattccagtactagaacgtctgcattgcaaaaccagaaatgtccagttctggtttggcacacagaagagattttcaagccttggcaactcctcagtgcaggcagtggaggaaatgctccagctttgtgaaaatcttctcccttcgctcctcatggagaataaccgttcatcactgcagccatgaatctgccacactgaatagatatggtctgaaaatctaggaacaaatggaatcactttggaaacctgtaaattttaaagccgaaagggcgctccagggaaccaactacatgaagttctctggaagccacaggatgggaggtttggtgggggtggaggtgaccattgaattttacataggcaaatgtaagacaatcttatccatagacagaaaaatccaaactcaccgagagtaaaaggatgacagttgaaataacagctaattgttattaagtatttgctaagttccaaagcacttttcatagattagctcaattaattctcacaacagccctgtaaggtagggaccatgatcatctccagaagaggctaaataaattgccaaagatcacatatgtagcaagtggtggcagagtgaggattcatacctggacagcttaactttaaagccatggtgctaatgcattcattcattcatccatccattcattcatccatccattcattcaatgattatattggatgcctactacatgctaagaactatgttcaaggtgctgaactacagctgtgaacaatgcaagtacaaatacatgaagcagagttggggagaaagtaagctggagaaataagacataaactaggttagatggtgaacatgattaaggaaaaaactagagcagggaggaaagagaaatgtgcagggggaaggtcaattctgagtgagccaacaggcaaggtctcactaagaaagcggcattaaataaagatttgaggaagtgagggagtaaaccttgcagctatctaaaaggagagcatgagaggtagaggagacagcaaatacaaagaccctgaagcaggggcaaggctaatgagttctagaattagcaaggagtccagtgtaacttgaacagagtgatcaaagaacagagcaggaggaaatgaggtcagagaggtaaacaggctggagtgcagtggcaccgtctcggctcactgcaacctccacctcccgggttcaagcgattctcctgcctcagtctccctagtagctgggattacaggcatgcaccaccacacccggctaatttttgtatttttagtaaagacggtgtttcaccatgttggccaggctggtcttgaactcctgacctcaagtgatccacccgcctcaacctcccaaagtgctgggattacaggcgtgagccaccgcgcccggccaggattcttttaatcaagtatgatgcagccatcagggttggagcattctaggaagagggaacagcatgcaccaaggcaacaggaaagcacataaaaaaggagttgctggaagattcatttcaacccactatcaagtaaatattaaacttactgtataaaaatgttagggccaggaggggtggctcacgcctgtaaacccggcactttgagaggccgaggcaggaggatcggttgaacccaggagttcgagaccagcctgaccaacagggcgaaaccccgtctctactaaaaatacaaaaactagccgggcgtggtggtgggcgcctgtaatcccagcttctcgggaggctgaggcaggagaattgcttgaacctgggaggcggaggttgcagtgagctgagatcacgccctgcactccagcctgggcgacagagggagacttggtcatgctccctccccgccctccgtcagttttaggaataaatacctttttatttaagctaaagtgtgggtacacccttcctctaggattctccatcaaggaataagaagccatattaggacaatttagagggcagttaaccctagtagacatagtggttcttaaaaggcttggggcctcagactgtacacaggcttcacatggaatctgatttgttcctttatcccagtccctcacccagaacccgaatctagcccttcatgttataaaaagggccagaggtccaaagagggtaagtgccttgtgcaaaattattcaactactttgtagaggattttaactagggttcagttagctccgcccacactataaaaggctctttctaaaaaacgaaacatgattaagggcacacggctccagcgttaacaaagctctttgttagctgggaaatgcccccctccccgactcatctgcttatcatctgcatagaattatgtactcaaaagcaggaaaattattgagaaaaccattggtccccggctgcagaccccaaggttgggagctggttcccgtttcctcccagagccggcagggggggcaccaggcaaagtttgcaagacgcgcctccctcccacccctcccccttcctcgcccaacttcccatagccgcggcctcaactaaaagtggccattgacctttcaagctttcgagcagtgatgcaatagaatagtatttcaaagaaaaatgcttatcgaaattttggatccggttttcccgtgattgttaagggtttcttttaaaaagtaggtcacatttcaagtaggtcatatttcgggggcgggtgcgcagacaaggagatgagtttccactaaggccagggggcctccaacggggttggaggtgagaatcccaggtagggtagaggtgccgagatccttccgaatcccagccctggggcgtcagccctgcagggaatggcagagacactctccggactgagggaaccgaggccagtcaccaagccccttccgggcgcgcaggtaagggcgcccccttagcagccggcgcaggtgacccgggcgggccgccgggtctaccggagacgttggagcagaggggaggaggaagggaggagccgggtgggtgcgggtgacaaggagccggagcgccagggggaggggactaaggacggccggcgccgcttaaggaggcgctgctctcccgctcgctgccttccaggacctgatcaaggggaccgcctccggtccccggccgtgggcaccgggacgagcacggcgtccccacgccatcgatgtgtcttagagccggagagtctggtttccgaggacccacagtcgctcctgcacgcccaccccccgcaaaagtgcggccaggagggtcgcatcgagggggcgccgccgggatgtttagaggaacccacccccgtggcaggccaagggccaaggatcgctatccttccctgaacccgggcgctcagctggcccgggtagggggcaggctccggccgccgaaacggggttggctgtagccggtggccggggagatctctagcttgcgcccgagcaccccggggtgtggggccagaggcaggccgaccccggcgtgcacaccgcccgccctgcacccgagcgctctcacccggtcttccctgaagcctgtgtattgcgaccgagcctctttaaagcagtagcggggcccgcggtcacgtgaggccgattcctggaaagttcctggaaagccgcctccgcagcagccgggcggggcgcgagcggagcgctgactggggagggaggcggggagcaagggaggcgcgtcggtctgggaagtcgcgcgcactcgctgctcctgggaccgacgtttaactcttgccaagtctcgtcgcagccgccgcggctggcgggccttgggcttcccctgaagcatgagccctctcgcccgcagccaccctcaccgcgtggcccgcggacagtgcgcgccggggtcccgggtgcacagcctcaggataccccgtgcccgcagctcgggcgcccgcggcaggtaccggtagtgggggaagcccgaaggctccgccccgaggagagttaccggggagggcggcgaggcgcggccgcgtgcgccggggagcggcggacggcctgggcttccgcagctcggagcgccggggaagagagagtccgaaccgcggctctgccccgcggcctagcgttgctgcctgctttctccagccccttctcggcgtctggaagtgtctggagtttcttttttttatttcccctaaactgccattcaaattaataatcctcctaataacctgatctcccgctcctccccaccggcctgcctcccgccctcgctccttcctccctccctccctctctccttcccacctccagggtcgcagccggagggaaacccggcagcagtccgagagtggaggtgtcccagcccgtagggggcgtcgccgcgcggtgggggatggggtcgaggcaatgatcctcgaggcttttgtgtgccctctgcgcacggaactccgaccgccgcctccgagcgcggggctggttgcatccccgggcatctcgttcccaaattaaacgttaacgggggaaacaagggcagacgcccctcctctcccgggcccctcccacctccccttttccccccacccccccgccccatgtccgctgaggaggctgcctggtgcggaggcggcggcggcggccgcggccgaggtcgaggtaagagcgcggcgttggtggtttgcattttccgcagcgc

SEQ ID NO: 4 - DNA, 597 bp, Homo sapiens
  CDS (1)..(597)
atgaccctgcggtgccttgagccctccgggaatggcggggaagggacgcggagccagtgggggaccgcggggtcggcggaggagccatccccgcaggcggcgcgtctggcgaaggccctgcgggagctcggtcagacaggatggtactggggaagtatgactgttaatgaagccaaagagaaattaaaagaggcaccagaaggaactttcttgattagagatagctcgcattcagactacctactaacaatatctgttaaaacatcagctggaccaactaatcttcgaatcgaataccaagacggaaaattcagattggactctatcatatgtgtcaaatccaagcttaaacaatttgacagtgtggttcatctgatcgactactatgttcagatgtgcaaggataagcggacaggtccagaagccccccggaacggcactgttcacctttatctgaccaaaccgctctacacgtcagcaccatctctgcagcatctctgtaggctcaccattaacaaatgtaccggtgccatctggggactgcctttaccaacaagactaaaagattacttggaagaatataaattccaggtataa

SEQ ID NO: 5 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

SEQ ID NO: 6 - DNA, 39 bp, Artificial Sequence
  chemically-synthesized primer sequence
cggggtaccgccatgaccctgcggtgcctggagccctcc

SEQ ID NO: 7 - DNA, 36 bp, Artificial Sequence
  chemically-synthesized primer sequence
ccgctcgagttatacctggaatttatattcttccaa

SEQ ID NO: 8 - DNA, 36 bp, Artificial Sequence
  chemically-synthesized primer sequence
gtgacgcgtgctccctccaagtggtggaaaagttga

SEQ ID NO: 9 - DNA, 36 bp, Artificial Sequence
  chemically-synthesized primer sequence
gtggctagcgcgctgcggaaaatgcaaaccaccaac

SEQ ID NO: 10 - DNA, 36 bp, Artificial Sequence
  chemically-synthesized primer sequence
gtgacgcgtgacctgtatggtcattatcactcatca

SEQ ID NO: 11 - DNA, 35 bp, Artificial Sequence
  chemically-synthesized primer sequence
gtggctagcgcgctcttacctcgacctcggccgcg

SEQ ID NO: 12 - DNA, 24 bp, Artificial Sequence
  chemically-synthesized primer sequence
cctttatctgaccaaaccgctcta

SEQ ID NO: 13 - DNA, 25 bp, Artificial Sequence
  chemically-synthesized primer sequence
tgttaatggtgagcctacagagatg

SEQ ID NO: 14 - DNA, 21 bp, Artificial Sequence
  chemically-synthesized primer sequence
cctgacctgccgtctagaaaa

SEQ ID NO: 15 - DNA, 18 bp, Artificial Sequence
  chemically-synthesized primer sequence
cgcctgcttcaccacctt